(19) United States
(12) Patent Application Publication
(10) Pub. No.: US 2020/0317708 A1
(43) Pub. Date: Oct. 8, 2020

(54) SUGAR-LINKED AMINO ACIDS FOR SOLID-PHASE PEPTIDE SYNTHESIS

(71) Applicant: INDIANA UNIVERSITY RESEARCH AND TECHNOLOGY CORPORATION, Indianapolis, IN (US)

(72) Inventors: Nicola L. POHL, Bloomington, IN (US); Ravi Kumar HITTANAHALLI KOPPAL VEERA, Bloomington, IN (US)

(21) Appl. No.: 16/753,998

(22) PCT Filed: Oct. 8, 2018
(86) PCT No.: PCT/US2018/054837

§ 371 Date: Apr. 6, 2020

Related U.S. Application Data
(60) Provisional application No. 62/570,067, filed on Oct. 9, 2017.

(51) Int. Cl. C07H 5/10; C07H 1/00

(52) U.S. Cl. CPC C07H 5/10; C07K 1/04; C07H 1/00

(57) ABSTRACT

The present disclosure relates to sugar-linked amino acids and processes for preparing the same. The sugar-linked amino acids may be used for solid-phase peptide synthesis. A sugar compound and an amino acid compound having a nucleophilic side chain are reacted in a heated halogenated solvent. The reaction is catalyst by a Lewis acid, such as InBr₃. The reaction is performed as a batch or continuous process.

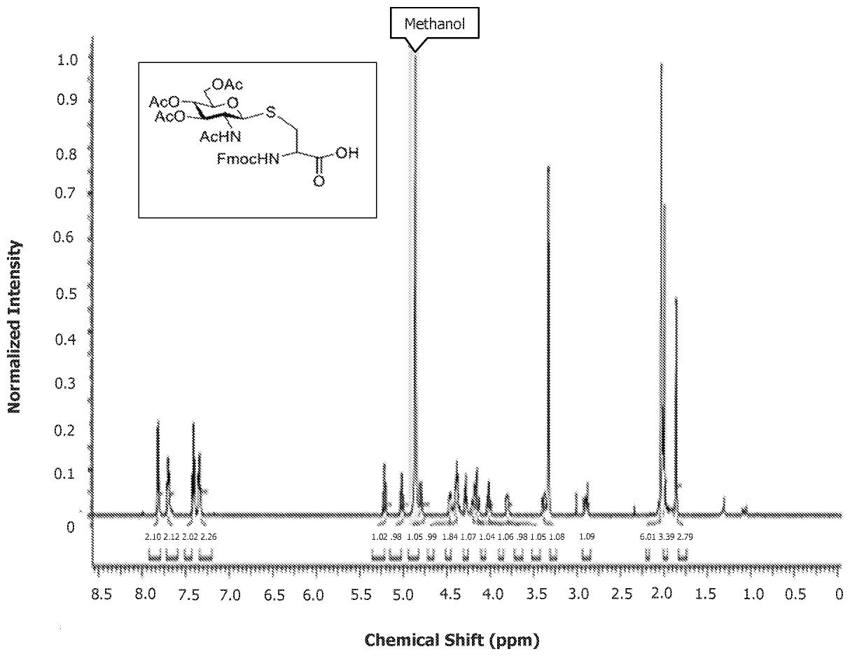

### CROSS-REFERENCE TO RELATED APPLICATIONS

This application claims the benefit of U.S. Provisional Patent Application No. 62/570,067, filed Oct. 9, 2017, the disclosure of which is hereby incorporated by reference in its entirety.

### FIELD

The present disclosure relates to sugar-linked amino acids and processes for preparing the same. Specifically, this disclosure relates to sugar-linked amino acids for solid-phase peptide synthesis and processes for preparing the same.

### BACKGROUND

Sugar-linked amino acids for solid-phase synthesis are expensive to prepare and may sell for sell for $10,000/gram, commercially. This cost creates a hurdle for companies and academic and industry labs that want to explore the effects of modified peptides. Thus, there is a need in the art for less expensive methods of preparing modified peptides, such as sugar-linked amino acids.

### SUMMARY

The present disclosure provides sugar-linked amino acids and processes for preparing the same. In some embodiments, the sugar-linked amino acids are used for solid-phase peptide synthesis.

In some embodiments, the present disclosure provides a process for preparing sugar-linked amino acid of the formula I

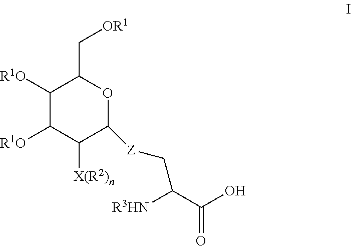

wherein

X is O or N;

each R¹ is independently a hydroxyl protecting group;

each R² is H or a protecting group; provided that when X is O, R² is a protecting group; and when X is N, at least one R² is a protecting group;

R³ is an N-terminal protecting group;

Z is O or S; and

n is 1 when X is O and n is 2 when X is N;

the process comprising contacting a compound of the formula II

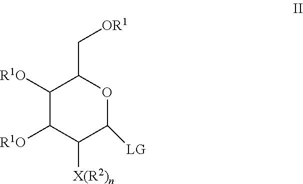

wherein

each R¹ is independently a hydroxyl protecting group;

X is O or N;

each R² is H or a protecting group; provided that when X is O, R² is a protecting group; and when X is N, at least one R² is a protecting group;

LG is a leaving group;

n is 1 when X is O and n is 2 when X is N;

with a compound of the formula III

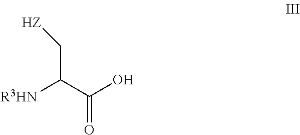

wherein

R³ is an N-terminal protecting group and

Z is O or S;

in the presence of a Lewis acid catalyst.

In some embodiments, the present disclosure provides a process for preparing sugar-linked amino acid of the formula IV

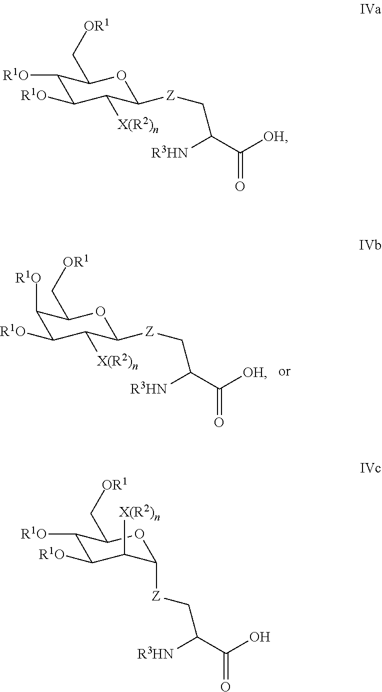

wherein

each R¹ is independently a hydroxyl protecting group;

X is O or N;

each R² is H or a protecting group; provided that when X is O, R² is a protecting group; and when X is N, at least one R² is a protecting group;

R³ is an N-terminal protecting group;

Z is O or S; and

n is 1 when X is O and n is 2 when X is N;

the process comprising contacting a compound of the formula V

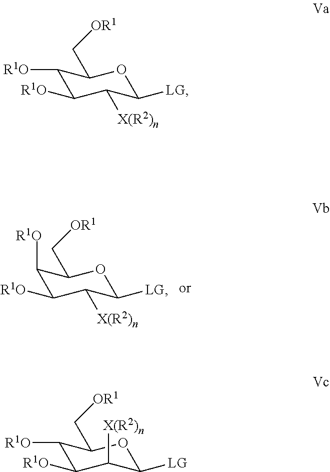

wherein

each R¹ is independently a hydroxyl protecting group;

X is O or N;

each R² is H or a protecting group; provided that when X is O, R² is a protecting group; and when X is N, at least one R² is a protecting group;

LG is a leaving group; and

n is 1 when X is O and n is 2 when X is N;

with a compound of the formula VI

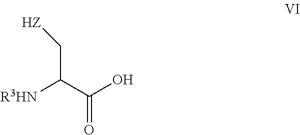

wherein

R³ is an N-terminal protecting group and

Z is O or S;

in the presence of a Lewis acid catalyst.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A process for preparing a sugar-linked amino acid of the formula I

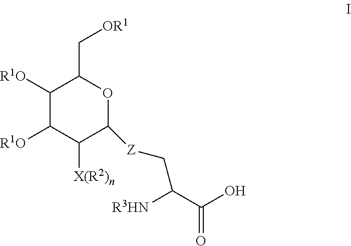

wherein

R¹ is a hydroxyl protecting group;

X is O or N;

each R² is H or a protecting group; provided that when X is O, R² is a protecting group; and when X is N, at least one R² is a protecting group;

Z is O or S; and

n is 1 when X is O and n is 2 when X is N;

the process comprising contacting a compound of the formula II

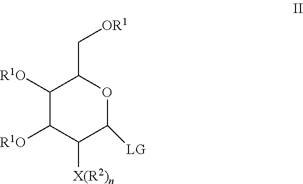

wherein

R¹ is a hydroxyl protecting group;

X is O or N;

each R² is H or a protecting group; provided that when X is O, R² is a protecting group; and when X is N, at least one R² is a protecting group;

LG is a leaving group; and

n is 1 when X is O and n is 2 when X is N;

with a compound of the formula III

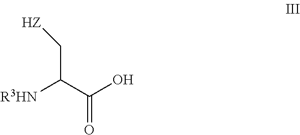

wherein

R³ is an N-terminal protecting group and

Z is O or S;

in the presence of a Lewis acid catalyst.

2. The process of clause 1, wherein X is N and one R² is hydrogen.

3. The process of clause 1 or 2, wherein Z is S.

4. The process of any one of the preceding clauses, wherein each R¹ is acetyl.

5. The process of any one of the preceding clauses, wherein one R² is acetyl.

6. The process of any one of the preceding clauses, wherein R³ is Fmoc or Boc.

7. The process of any one of the preceding clauses, wherein R³ is Fmoc.

8. The process of any one of the preceding clauses, wherein the Lewis acid catalyst comprises indium.

9. The process of any one of the preceding clauses, wherein the Lewis acid catalyst is InBr₃.

10. The process of any one of the preceding clauses, wherein the compound of the formula II and the compound of the formula III are combined in a halogenated solvent.

11. The process of any one of the preceding clauses, wherein the halogenated solvent in chloroform.

12. The process of any one of the preceding clauses, further comprising heating a reaction mixture of the compound of the formula II and the compound of the formula III.

13. The process of clause 12, wherein the reaction mixture is heated for at least 1 hour.

14. The process of clause 12 or 13, wherein the reaction mixture is heated for about 15 to about 22 hours.

15. The process of any one of clauses 1 to 12, wherein a reaction mixture of the compound formula II and the compound of the formula III are contacted in a continuous flow process.

16. The process of clause 15, wherein the reaction mixture is pumped at a flow rate of about 100 μL/min to about 300 μL/min.

17. The process of clause 15 or 16, wherein the reaction mixture is pumped at a flow rate of about 200 μL/min.

18. The process of any one of clauses 15 to 17, wherein the reaction mixture is pumped with residence time of about 1 min to about 5 min.

19. The process of any one of clauses 15 to 18, wherein the reaction mixture is pumped with residence time of about 2.5 min.

20. The process of any one of the preceding clauses, wherein two equivalents of the compound formula II and one equivalent of the compound of the formula III are combined.

21. A process for forming a sugar-linked polypeptide, the method comprising preparing one or more sugar-linked amino acids according to the process of any one of the preceding clauses and deprotecting the one or more sugar-linked amino acids by removing R³.

22. A process for preparing a sugar-linked amino acid of the formula IV

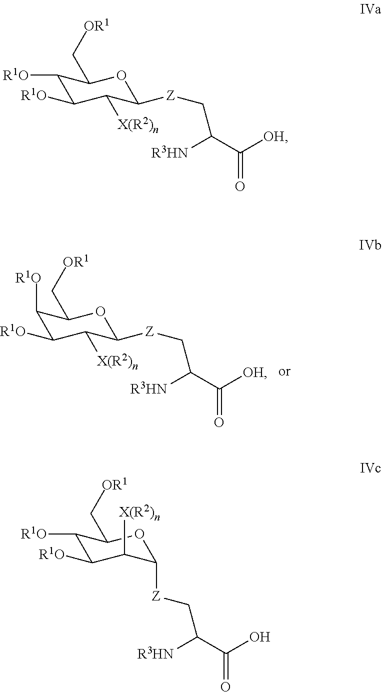

wherein

each R¹ is independently a hydroxyl protecting group;

X is O or N;

each R² is H or a protecting group; provided that when X is O, R² is a protecting group; and when X is N, at least one R² is a protecting group;

R³ is an N-terminal protecting group;

Z is O or S; and

n is 1 when X is O and n is 2 when X is N;

the process comprising contacting a compound of the formula V

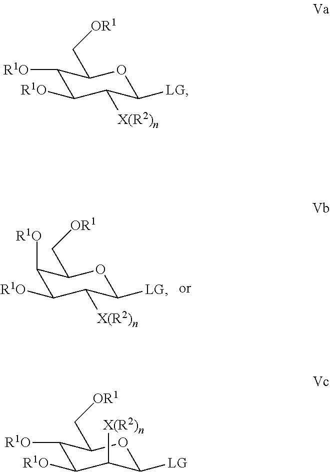

wherein

each R¹ is independently a hydroxyl protecting group;

X is O or N;

each R² is H or a protecting group; provided that when X is O, R² is a protecting group; and when X is N, at least one R² is a protecting group;

LG is a leaving group; and

n is 1 when X is O and n is 2 when X is N;

with a compound of the formula VI

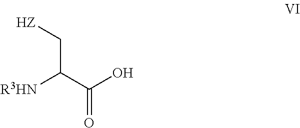

wherein

R³ is an N-terminal protecting group and

Z is O or S;

in the presence of a Lewis acid catalyst.

23. The process of clause 22, wherein X is N and one R² is hydrogen.

24. The process of clause 22 or 23, wherein Z is S.

25. The process of any one of clauses 22 to 24, wherein R¹ is acetyl.

26. The process of any one of clauses 22 to 25, wherein one R² is acetyl.

27. The process of any one of clauses 22 to 26, wherein R³ is Fmoc or Boc.

28. The process of any one of clauses 22 to 27, wherein R³ is Fmoc.

29. The process of any one of clauses 22 to 28, wherein the Lewis acid catalyst comprises indium.

30. The process of any one of clauses 22 to 29, wherein the Lewis acid catalyst is InBr₃.

31. The process of any one of clauses 22 to 30, wherein the compound of the formula V and the compound of the formula VI are combined in a halogenated solvent.

32. The process of any one of clauses 22 to 31, wherein the halogenated solvent in chloroform.

33. The process of any one of clauses 22 to 32, further comprising heating a reaction mixture of the compound of the formula V and the compound of the formula VI.

34. The process of clause 33, wherein the reaction mixture is heated for at least 1 hour.

35. The process of clause 33 or 34, wherein the reaction mixture is heated for about 15 to about 22 hours.

36. The process of any one of clauses 22 to 33, wherein a reaction mixture of the compound formula V and the compound of the formula VI are contacted in a continuous flow process.

37. The process of clause 36, wherein the reaction mixture is pumped at a flow rate of about 100 μL/min to about 300 μL/min.

38. The process of clause 36 or 37, wherein the reaction mixture is pumped at a flow rate of about 200 μL/min.

39. The process of any one of clauses 36 to 38, wherein the reaction mixture is pumped with residence time of about 1 min to about 5 min.

40. The process of any one of clauses 36 to 39, wherein the reaction mixture is pumped with residence time of about 2.5 min.

41. The process of any one of clauses 22 to 40, wherein two equivalents of the compound formula V and one equivalent of the compound of the formula VI are combined.

42. A process for forming a sugar-linked polypeptide, the method comprising preparing one or more sugar-linked amino acids according to the process of any one of clauses 22 to 41 and deprotecting the one or more sugar-linked amino acids by removing R³.

43. A sugar-linked amino acid of the formula VII

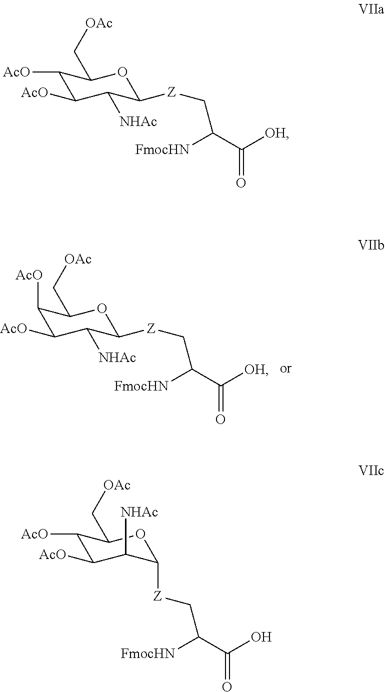

wherein Z is O or S.

44. The compound of any of the preceding clauses, wherein Z is S.

43. The process of any of the preceding clauses, wherein the compound of formula IV is prepared at a yield of at least about 50%, at least about 60%, at least about 70%, at least about 80%, or at least about 85%.

44. A sugar-linked amino acid of the formula VII

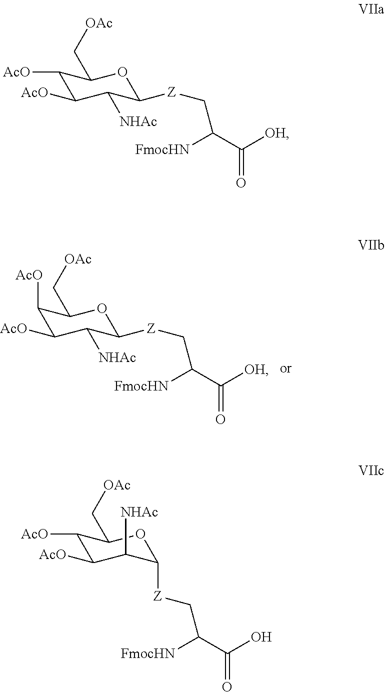

wherein Z is O or S.

45. The compound of any of the preceding clauses, wherein Z is S.

46. A sugar-linked amino acid of the formula VIII

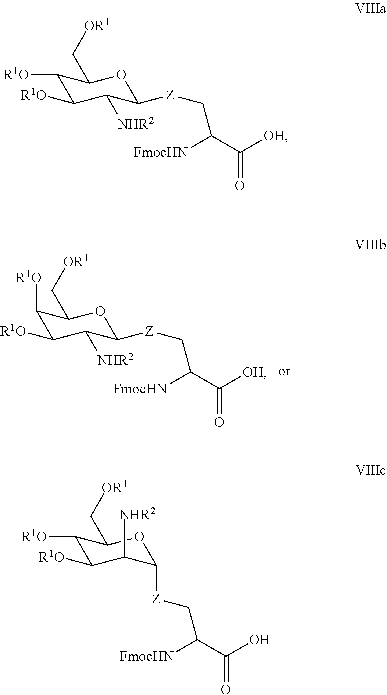

wherein

R¹ is a hydroxyl protecting group;

Z is O or S; and

each R² is a protecting group.

47. The compound any of the preceding clauses, wherein Z is S.

48. The compound any of the preceding clauses, wherein each R¹ and R² is independently RC(O)—, wherein R is alkyl, alkenyl, or aryl, and wherein each hydrogen atom on aryl, alkenyl, or aryl is optionally substituted with halo.

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O ), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, including an all-carbon 5-member/6-member or 6-member/6-member fused bicyclic ring, or a multicyclic fused ring (a “fused” ring system means that each ring in the system shares an adjacent pair of carbon atoms with each other ring in the system) group, where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₉, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like. Illustrative examples of cycloalkyl groups shown in graphical representations include the following entities, in the form of properly bonded moieties:

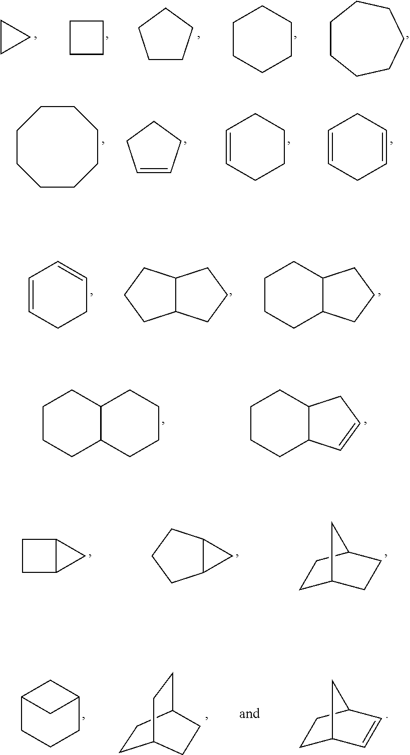

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1,2,3,4-tetrahydropyridinyl, and the like. Illustrative examples of heterocycloalkyl groups shown in graphical representations include the following entities, in the form of properly bonded moieties:

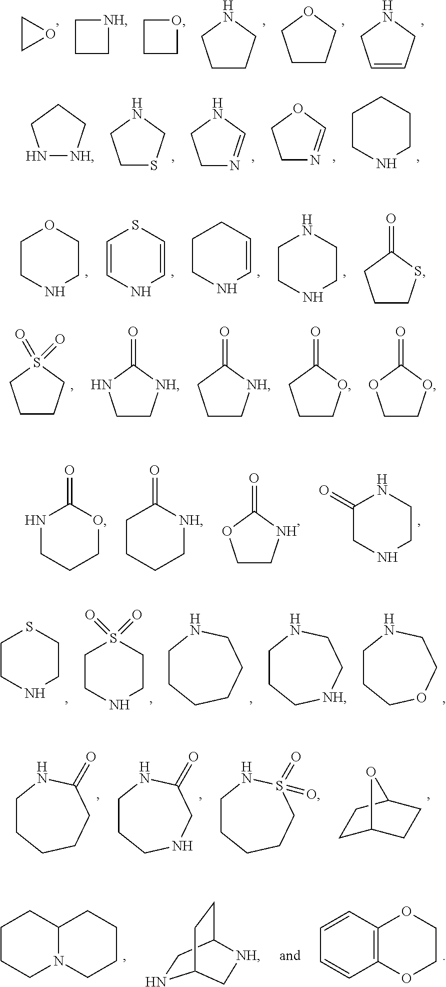

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like. Illustrative examples of heteroaryl groups shown in graphical representations, include the following entities, in the form of properly bonded moieties:

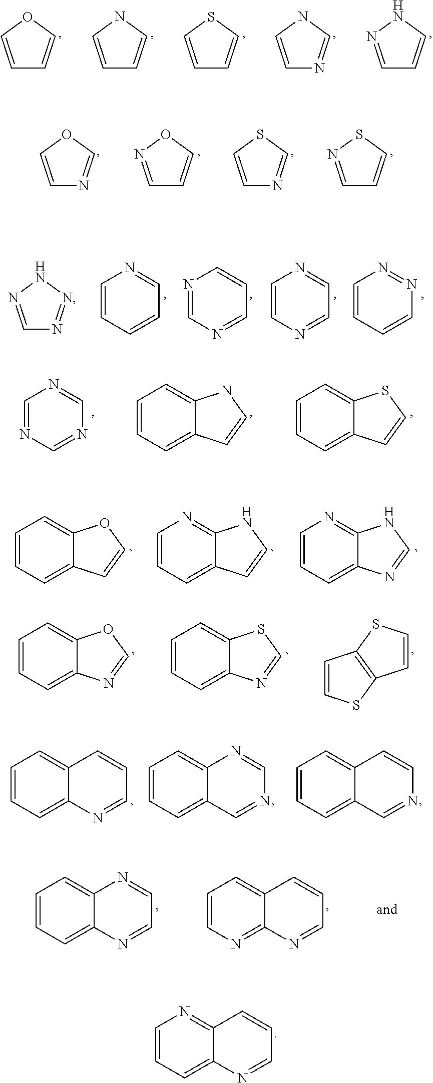

### BRIEF DESCRIPTION OF THE DRAWING

The FIGURE shows an NMR spectrum of the compound prepared in Example 1.

### DETAILED DESCRIPTION

The present disclosure is directed to processes for preparing certain sugar-linked amino acids for solid-phase peptide synthesis. It will be understood that in some embodiments, the present disclosure provides processes for preparing sugar-linked serine and threonine compounds for solid-phase peptide synthesis. These compounds may be used as building blocks related to glycosylated serine, threonine, or cysteine derivatives. The processes, which may be accomplished using a batch process of continuously flow chemistry, can be represented the following general Scheme A.

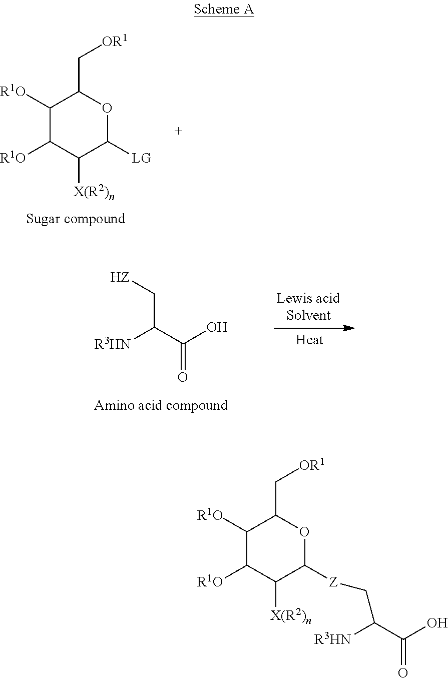

wherein each of R¹, R², R³, X, Z, LG, and n is as defined herein. In some embodiments, the sugar compound may be a glucose, a mannose, a galactose, or a derivative, such as a C-2 amino, thereof. In some embodiments, the amino acid compound may be a cysteine, a threonine, or a serine.

In some embodiments, X is O or N. In some embodiments, X is O. In some embodiments, X is N.

In some embodiments, each R² is H or a protecting group. Illustratively, if X is O, the O may be substituted by an acetyl group, a trifluoroacetyl group, a trimethylacetyl group, or a benzoyl group, although other substitutions are contemplated. Additional protecting groups suitable for use here are described in Greene's Protective Groups in Organic Synthesis, 4th ed., published by John Wiley and Sons in 2007, which is hereby incorporated by reference in its entirety.

In some embodiments, when X is N, at least one R² is a protecting group. In some embodiments, when X is N, one R² is a protecting group and the other R² is H. In some embodiments, when X is N, each R² is independently a protecting group. In some embodiments, when X is N, two R² groups can combine to form a single protecting groups, such as a phthalate group. Additional protecting groups suitable for use here are described in Greene's Protective Groups in Organic Synthesis, 4th ed., published by John Wiley and Sons in 2007, which is hereby incorporated by reference in its entirety.

In some embodiments, Z is O or S. In some embodiments, Z is O. In some embodiments, Z is S.

In some embodiments, n is an integer. In some embodiments, n may be 1 or 2. In some embodiments, n is 1 when X is O. In some embodiments, n is 2 when X is N.

More particularly, the processes of the present disclosure can be described by the following Scheme B.

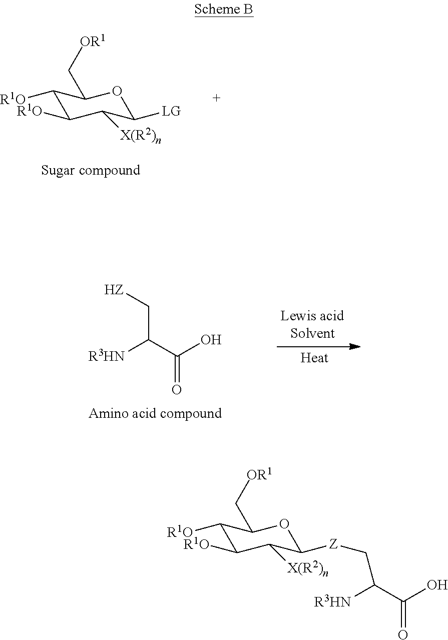

wherein each of R¹, R², R³, X, Z, LG, and n is as defined herein. Although a glucose derivative is shown as the sugar compound, Scheme B is equally applicable for other sugars such as mannose, galactose, and derivatives thereof. In some embodiments, the amino acid compound may be a cysteine, a threonine, or a serine.

It will be appreciated that the reaction can be carried out according to any of the conditions described herein.

In some embodiments, the sugar compound is any sugar capable of reacting with any amino acid side chain. The sugar is derivatized to include at least one leaving group. For example, the sugar compound may be glucose or glucosamine, derivatized to include a leaving group. In some embodiments, the sugar compound may be mannose or mannosamine, derivatized to include a leaving group. In some embodiments, the sugar compound may be galactose or galactosamine, derivatized to include a leaving group. The sugar may have any physically stable stereochemistry, including natural or unnatural configurations.

The leaving group may be any leaving group known to those skilled in the art that can be displaced via an SN1 or an SN2 process. In some embodiments, a hydroxyl group of the sugar is acylated with an acyl group of the formula RCO—, where R is an alkyl, alkenyl, or aryl group, wherein each hydrogen on the alkyl, alkenyl, or aryl group is optionally substituted, to form an acyloxy leaving group. In some embodiments, the hydroxyl group is substituted by an acetyl group or a trifluoroacetyl group, although other substitutions are contemplated. In some embodiments, the leaving group is on the anomeric carbon.

The other alcohols, amines, or similar groups on the sugar may also be functionalized. For example, the groups may by functionalized by reaction with an acyl group of the formula RCO—, where R is an alkyl, alkenyl group, or aryl group, wherein each hydrogen on the alkyl, alkenyl, or aryl group is optionally substituted, to form an acyloxy leaving group. In some embodiments, the optional substitution on the alkyl, alkenyl, or aryl group is halo. In some embodiments, the hydroxyl group is substituted by an acetyl group, a trifluoroacetyl group, a trimethylacetyl group, or a benzoyl group, although other substitutions are contemplated. Additional protecting groups suitable for use here are described in Greene's Protective Groups in Organic Synthesis, 4th ed., published by John Wiley and Sons in 2007, which is hereby incorporated by reference in its entirety.

The amino acid compound is any amino acid having a nucleophilic side chain. The amino acid compound may be a natural or unnatural amino acid. In some embodiments, the amino acid has a thiol or hydroxyl side chain. The amino acid compound may be serine or threonine. In some embodiments, the amino acid is cysteine.

It is to be understood that “amino acid compound” refers to both protected and unprotected amino acids that are capable of undergoing the reactions described herein. In some embodiments, the amino acid compound has a free carboxyl group in a C-terminus position and a protected amino group in an N-terminus position. The amino group is protected by any group useful in solid phase peptide synthesis (SPPS), such as a fluorenylmethyloxycarbonyl (Fmoc) group or a tert-butyloxycarbonyl (Boc) group. It is contemplated that after reaction of the sugar compound and the amino acid compound, the amino protecting group may be cleaved such that the amino group is able to react with a free carboxyl group on another amino acid to form a peptide bond. In some embodiments, the carboxyl group on the other amino acid is activated according to traditional peptide chemistry. Additional protecting groups suitable for use here are described in Greene's Protective Groups in Organic Synthesis, 4th ed., published by John Wiley and Sons in 2007, which is hereby incorporated by reference in its entirety.

The reaction between the sugar compound and the amino acid compound takes place in the presence of a Lewis acid catalyst. It is contemplated that the Lewis acid catalyst may be a Lewis acid catalyst useful in any nucleophilic substitution reaction described herein. In some embodiments, the Lewis acid catalyst is an indium catalyst, such as an indium (III) catalyst. Illustratively, the indium catalyst may be InBr₃. In some embodiments, the amount of Lewis acid present is sub-stoichiometric.

The reactions described herein may take place in any suitable solvent. In some embodiments, the solvent is a halogenated solvent. For example, the solvent may be dichloromethane (CH₂Cl₂) or chloroform (CHCl₃). In some embodiments, the solvent comprises a non-halogenated. In some embodiments, the non-halogenated solvent is acetonitrile or ethyl ether. In some embodiments, the solvent comprises a halogenated solvent and a non-halogenated solvent. In some embodiments, the solvent is a mixture of chloroform and acetonitrile. In some embodiments, the solvent is a mixture of chloroform and ethyl ether.

The reactions between the sugar compound and the amino acid compound described herein preferably take place at elevated temperatures. The temperature may be about 30° C. to about 150° C., about 50° C. to about 125° C., about 50° C. to about 100° C., about 70° C. to about 100° C., or about 75° C. to about 95° C. In some embodiments, the temperature may be the boiling temperature of the solvent in which the reaction takes place.

When the reaction occurs in a bath process, the reaction may occur over about 5 hours to about 30 hours, about 10 hours to about 30 hours, about 15 hours to about 30 hours, about 5 hours to about 25 hours, about 10 hours to about 25 hours, about 15 hours to about 25 hours, or about 15 hours to about 22 hours.

When the reaction occurs in a continuous process, the reaction may occur with a residence time from about 30 seconds to about 5 minutes, about 30 seconds to about 4 minutes, about 30 seconds to about 3 minutes, about 1 minute to about 5 minutes, about 1 minute to about 4 minutes, about 1 minute to about 3 minutes, about 2 minutes to about 5 minutes, about 2 minutes to about 4 minutes, about 2 minutes to about 3 minutes, or about 2.5 minutes. The flow rate may be about 50 μL/min to about 500 μL/min, about 100 μL/min to about 500 μL/min, about 200 μL/min to about 500 μL/min, about 50 μL/min to about 400 μL/min, about 100 μL/min to about 400 μL/min, about 200 μL/min to about 400 μL/min, about 50 μL/min to about 300 μL/min, about 100 μL/min to about 300 μL/min, about 200 μL/min to about 300 μL/min, or about 200 μL/min.

The process described herein provides the sugar-amino acid compound (compound formula VII) in a particular yield. In some embodiments, the yield is at least 30%, at least 40%, at least 50%, at least 70%, at least 80%, or at least 85%.

According to one aspect, a sugar-linked amino acid may have the formula VIII

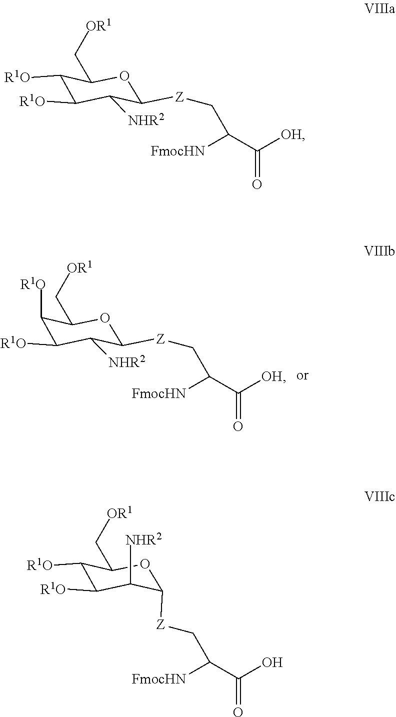

wherein each R¹ is independently a hydroxyl protecting group; Z is O or S; and each R² is a protecting group. In some embodiments, Z is S. In some embodiments, each R¹ and R² is independently RC(O)—, wherein R is alkyl, alkenyl, or aryl, and wherein each hydrogen atom on aryl, alkenyl, or aryl is optionally substituted. In some embodiments, the optional substitution is halo.

According to another aspect a sugar-linked amino acid of the formula VII

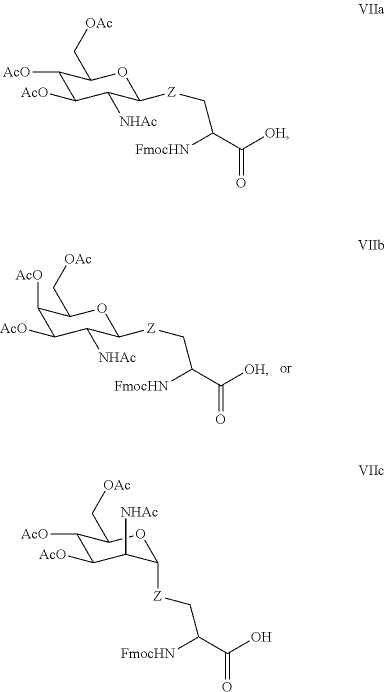

wherein Z is O or S.

### EXAMPLES

### Example 1

### Manual Batch Synthesis of AcGlcNAc-S-Cys

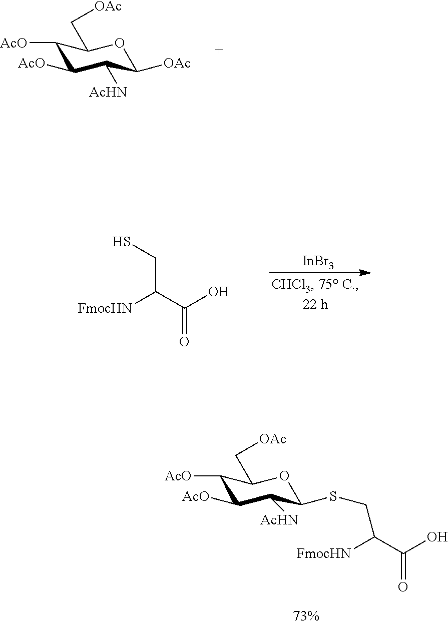

Peracetylated GlcNAc (2.0 eq) and Fmoc-Cys (1.0 eq) were taken up in chloroform to which was added indium bromide (0.5 eq). The mixture was refluxed for 15-22 hours. Upon completion of the reaction as monitored by thin layer chromatography, the reaction mixture was evaporated and purified using silica gel chromatography. The yield of AcGlcNAc-S-Cys was about 73%. The ¹H NMR is shown in the FIGURE.

### Example 2

### Continuous Flow Production of AcGlcNAc-S-Cys

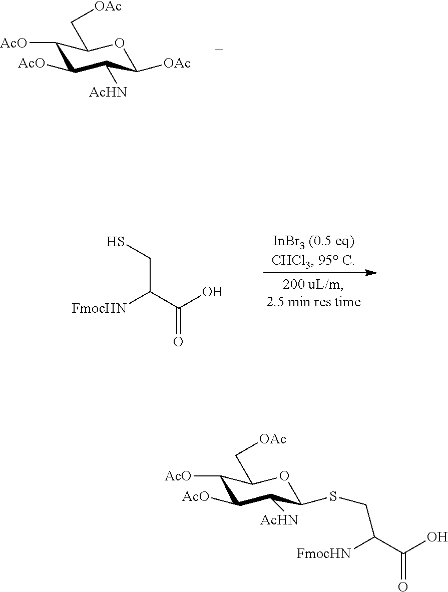

All starting materials including indium bromide were dissolved in chloroform and the solution was pumped through tubing with a 200 μL/min flow rate and 2.5 min residence time at elevated temperature and pressure. Data show at least 30% conversion to the desired product under these conditions.

### Example 3

### Synthesis of N^{α}-Fluoren-9-ylmethoxycarbonyl-S-(3,4,6-tetra-O-acetyl-2-acetamido-2-deoxy)-β-D-glucopyranosyl)-L-cysteine

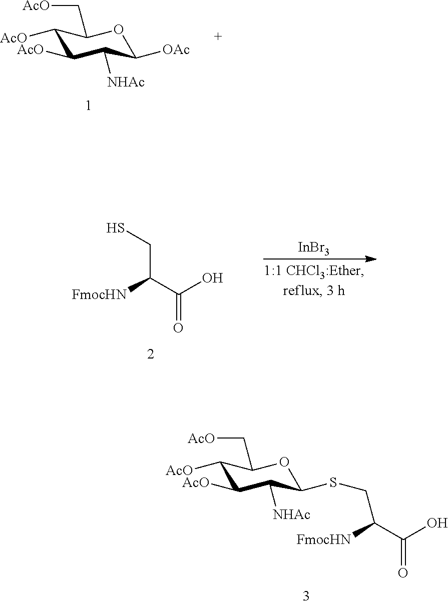

Peracetyl-β-D-GlcNAc (850 mg, 2.18 mmol, 1.5 eq), InBr₃ (232 mg, 0.65 mmol, 0.3 eq), and N^{α}-fluoren-9-ylmethoxycarbonyl-L-cysteine (500 mg, 1.47 mmol, 1 eq) were suspended in 1:1 CHCl₃:diethyl ether (30 mL). The reaction mixture was stirred at reflux for 3 hours. Gradually precipitation was observed and the precipitate was filtered and washed with diethyl ether. The crude was slurred with ether for 1 hr, filtered and washed with ether to afford 3 as an off-white powder (744 mg, 76%). R_{f} 0.4 (CH₂Cl₂/methanol, 9:1 v/v with 0.5% Acetic acid).

¹H NMR (500 MHz, Methanol-d₄) δ 7.82 (d, J=7.5 Hz, 2H), 7.70 (t, J=6.5 Hz, 2H), 7.41 (t, J=7.4 Hz, 2H), 7.34 (ddd, J=8.2, 6.8, 2.8 Hz, 2H), 5.22 (t, J=9.7 Hz, 1H), 5.01 (t, J=9.7 Hz, 1H), 4.80 (d, J=10.5 Hz, 1H), 4.46 (dd, J=8.9, 4.2 Hz, 1H), 4.40-4.36 (m, 2H), 4.27 (t, J=7.0 Hz, 1H), 4.22-4.11 (m, 2H), 4.02 (d, J=10.3 Hz, 1H), 3.84-3.73 (m, 1H), 3.38 (dd, J=14.3, 4.2 Hz, 1H), 2.94-2.85 (m, 1H), 2.03 (s, 2H), 2.02 (s, 3H), 2.00 (s, 3H), 1.86 (s, 2H).

¹³C NMR (126 MHz, Methanol-d4) δ 171.3, 171.1, 171.0, 169.5, 143.5, 141.1, 127.6, 127.0, 124.8, 119.8, 83.4, 75.6, 73.7, 68.4, 67.0, 62.1, 52.6, 52.6, 46.9, 30.5, 22.3, 20.3, 20.3, 20.3.

HRMS-ESI-TOF (m/z): Calcd. for [(C₃₂H₃₇N₂O₁₂S+]673.2067 (M+H⁺), Found 673.2067 (100%).

### Example 4

### Synthesis of N^{α}-Fluoren-9-ylmethoxycarbonyl-S-(3,4,6-tetra-O-acetyl-2-acetamido-2-deoxy)-β-D-galactopyranosyl)-L-cysteine

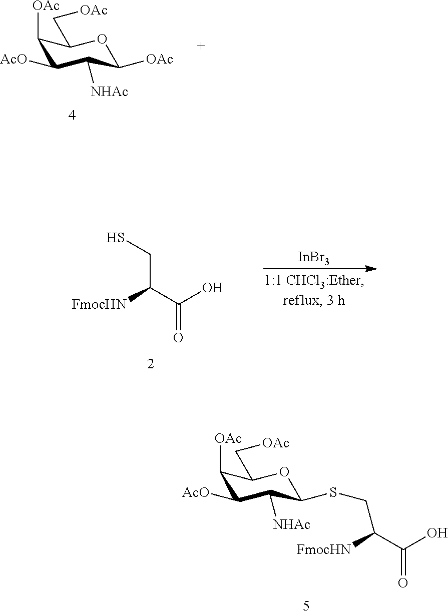

β-D-galactosamine pentaacetate (850 mg, 2.18 mmol, 1.5 eq), InBr₃ (232 mg, 0.65 mmol, 0.3 eq), and N^{α}-fluoren-9-ylmethoxycarbonyl-L-cysteine (500 mg, 1.47 mmol, 1 eq) were suspended in 1:1 CHCl₃:ethyl ether (30 mL). The reaction mixture was stirred at reflux for 3 hours. The solvent was evaporated and ether was added to the crude solid. The mixture was stirred for 1 hr at 0° C., filtered and washed with cold ether, Again the crude product was slurred with dichloromethane for 30 min and filtered and washed with cold DCM to afford 5 as an off-white powder (725 mg, 74%). R_{f} 0.4 (CH₂Cl₂/methanol, 9:1 v/v with 0.5% Acetic acid)

¹H NMR (500 MHz, Methanol-d₄) δ 7.81 (d, J=7.5 Hz, 2H), 7.71 (dd, J=18.5, 7.5 Hz, 2H), 7.45-7.39 (m, 2H), 7.38-7.31 (m, 2H), 5.37 (d, J=3.3 Hz, 1H), 5.07 (dd, J=10.8, 3.2 Hz, 1H), 4.72 (d, J=10.4 Hz, 1H), 4.47 (dd, J=9.4, 4.0 Hz, 1H), 4.43-4.32 (m, 2H), 4.32-4.24 (m, 2H), 4.17-4.07 (m, 2H), 3.98 (t, J=6.5 Hz, 1H), 3.45 (dd, J=14.4, 4.0 Hz, 1H), 2.88 (dd, J=14.4, 9.4 Hz, 1H), 2.12 (s, 3H), 1.98 (s, 2H), 1.97 (s, 3H), 1.88 (s, 2H).

¹³C NMR (126 MHz, Methanol-d₄) δ 172.19, 170.80, 170.66, 170.24, 157.03, 143.90, 143.75, 141.16, 127.42, 126.87, 126.83, 124.98, 124.85, 119.55, 119.53, 83.76, 74.34, 71.61, 66.99, 66.76, 61.74, 54.22, 48.48, 46.91, 31.04, 21.34, 19.16, 19.14, 19.13.

HRMS-ESI-TOF (m/z): Calcd. for [C₃₂H₃₇N₂O₁₂S+]673.2067 (M+H⁺), Found 673.2060 (100%).

### Example 5

### N^{α}-Fluoren-9-ylmethoxycarbonyl-S-(3,4,6-tetra-O-acetyl-2-acetamido-2-deoxy)-β-D-manopyranosyl)-L-cysteine

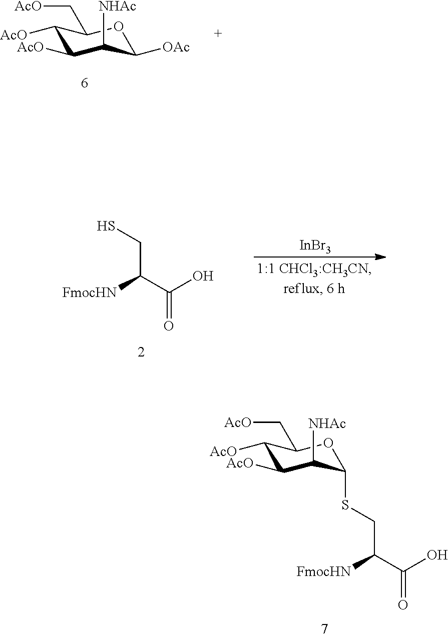

β-D-manosamine pentaacetate (850 mg, 2.18 mmol, 1.5 eq), InBr₃ (232 mg, 0.65 mmol, 0.3 eq), and N^{α}-fluoren-9-ylmethoxycarbonyl-L-cysteine (500 mg, 1.47 mmol, 1 eq) were suspended in CH₃CN:CHCl₃ (15 mL). The reaction mixture was stirred at reflux for 7 hours. The reaction mixture was concentrated in vacuo, and the residue was purified by flash chromatography ISCO (SiO₂, 0-1.0% of methanol in CH₂Cl₂ with 0.5% TFA) to afford 7 as an off-white powder (712 mg, 73%). R_{f} 0.4 (CH₂Cl₂/methanol, 9:1 v/v with 0.5% Acetic acid).

¹H NMR (500 MHz, Methanol-d₄) δ 7.80 (d, J=5.2 Hz, 2H), 7.69 (t, J=7.1 Hz, 2H), 7.39 (t, J=7.8 Hz, 2H), 7.32 (t, J=7.6, 5.3 Hz, 2H), 5.33 (s, 1H), 5.20 (dt, J=9.9, 2.6 Hz, 1H), 5.14 (dd, J=10.1, 4.6 Hz, 1H), 4.64 (s, 1H), 4.50-4.37 (m, 3H), 4.32-4.23 (m, 3H), 4.13 (dt, J=12.1, 2.6 Hz, 1H), 3.22-3.16 (m, 1H), 3.15-3.07 (m, 1H), 2.01 (s, 3H), 2.00 (s, 3H), 1.98 (s, 3H), 1.95 (s, 3H).

¹³C NMR (126 MHz, Methanol-d₄) δ 172.09, 171.14, 170.14, 170.12, 156.96, 143.82, 141.17, 127.42, 126.83, 126.83, 124.96, 124.86, 119.52, 84.79, 69.80, 69.18, 66.70, 66.40, 62.64, 54.40, 51.12, 48.10, 47.00, 33.72, 20.93, 19.33, 19.21, 19.17.

HRMS-ESI-TOF (m/z): Calcd. for [(C₃₂H₃₇N₂O₁₂S+] 673.2067 (M+H⁺), Found 673.2062 (100%).

1. A process for preparing a sugar-linked amino acid of the formula I 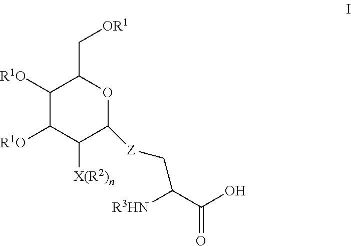
wherein
each R¹ is independently a hydroxyl protecting group;
X is O or N;
each R² is H or a protecting group; provided that when X is O, R² is a protecting group; and when X is N, at least one R² is a protecting group;
Z is O or S;
R³ is an N-terminal protecting group; and
n is 1 when X is O and n is 2 when X is N;
the process comprising contacting a compound of the formula II 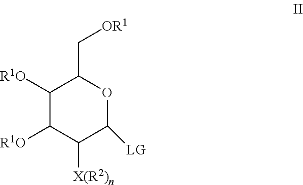
wherein
each R¹ is independently a hydroxyl protecting group;
X is O or N;
each R² is H or a protecting group; provided that when X is O, R² is a protecting group; and when X is N, at least one R² is a protecting group;
LG is a leaving group; and
n is 1 when X is O and n is 2 when X is N;
with a compound of the formula III 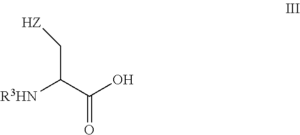
wherein
R³ is an N-terminal protecting group; and
Z is O or S;
in the presence of a Lewis acid catalyst.

2. The process of claim 1, wherein X is N and one R² is hydrogen.

3. The process of claim 1, wherein Z is S.

4. The process of claim 1, wherein each R¹ is acetyl.

5. The process of claim 1, wherein one R² is acetyl.

6. The process of claim 1, wherein R³ is Fmoc or Boc.

7. (canceled)

8. The process of claim 1, wherein the Lewis acid catalyst comprises indium.

9. The process of claim 1, wherein the Lewis acid catalyst is InBr₃.

10. (canceled)

11. (canceled)

12. The process of claim 1, further comprising heating a reaction mixture of the compound of the formula II and the compound of the formula III.

13.-14. (canceled)

15. The process of claim 1, wherein a reaction mixture of the compound formula II and the compound of the formula III are contacted in a continuous flow process.

16.-20. (canceled)

21. A process for forming a sugar-linked polypeptide, the process comprising deprotecting one or more sugar-linked amino acids of formula I 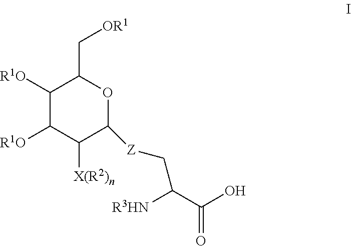
wherein
each R¹ is independently a hydroxyl protecting group;
X is O or N;
each R² is H or a protecting group; provided that when X is O, R² is a protecting group; and when X is N, at least one R² is a protecting group;
Z is O or S;
R³ is an N-terminal protecting group; and
n is 1 when X is O and n is 2 when X is N;
the process comprising removing R³.

22. The process of claim 1 wherein the compound of formula I is of formula IVa, IVb, or IVc 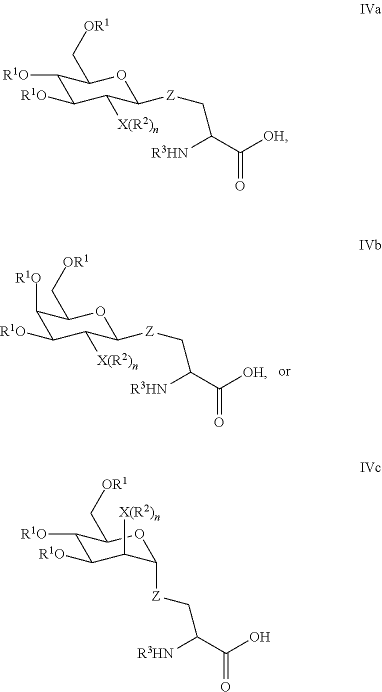
wherein
each R¹ is independently a hydroxyl protecting group;
X is O or N;
each R² is H or a protecting group; provided that when X is O, R² is a protecting group; and when X is N, at least one R² is a protecting group;
R³ is an N-terminal protecting group;
Z is O or S; and
n is 1 when X is O and n is 2 when X is N;
the process comprising contacting a compound of the formula Va, Vb, or Vc 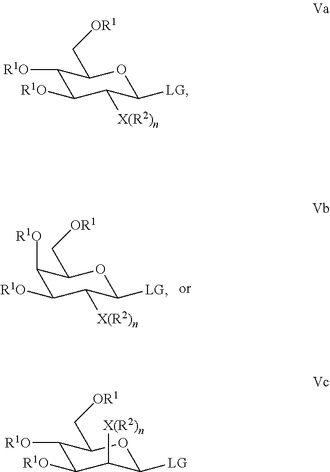
wherein
each R¹ is independently a hydroxyl protecting group;
X is O or N;
each R² is H or a protecting group; provided that when X is O, R² is a protecting group; and when X is N, at least one R² is a protecting group;
LG is a leaving group; and
n is 1 when X is O and n is 2 when X is N;
with a compound of the formula VI 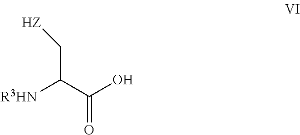
wherein
R³ is an N-terminal protecting group and
Z is O or S;
in the presence of a Lewis acid catalyst.

23.-28. (canceled)

29. The process of claim 22, wherein the Lewis acid catalyst comprises indium.

30. The process of claim 22, wherein the Lewis acid catalyst is InBr₃.

31.-33. (canceled)

34. The process of claim 22, further comprising heating a reaction mixture of the compound of the formula V and the compound of the formula VI.

35.-43. (canceled)

44. The sugar-linked amino acid of claim 46, having the structure of formula VIIa, VIIb, or VIIc, 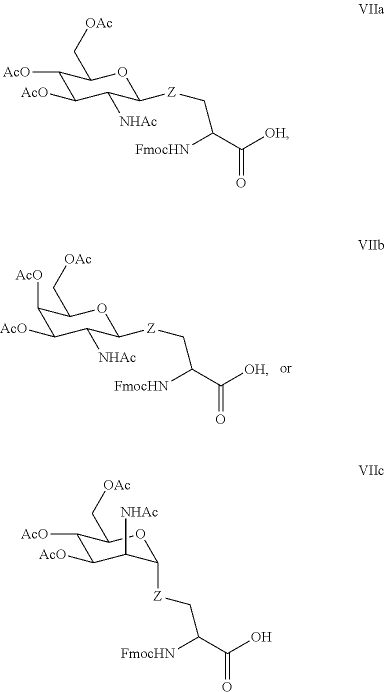
wherein Z is O or S.

45. The compound of claim 44, wherein Z is S.

46. A sugar-linked amino acid of the formula VIII 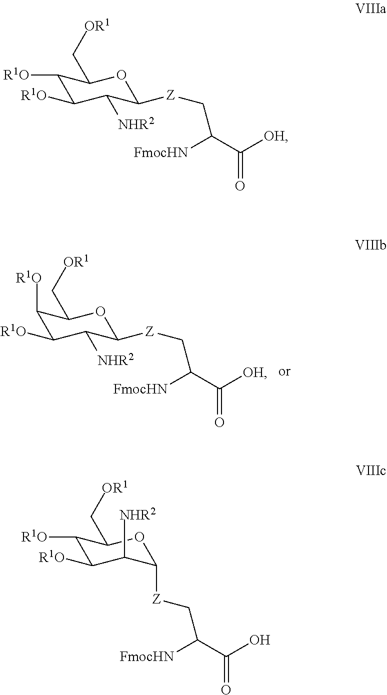
wherein
R¹ is a hydroxyl protecting group;
Z is O or S; and
each R² is a protecting group.

47. The compound of claim 46, wherein Z is S.

48. The compound of claim 46, wherein each R¹ and R² is independently RC(O)—, wherein R is alkyl, alkenyl, or aryl, and wherein each hydrogen atom on aryl, alkenyl, or aryl is optionally substituted with halo.
